(19) United States
(12) Patent Application Publication
(10) Pub. No.: US 2006/0000469 A1
(43) Pub. Date: Jan. 5, 2006

(54) Nebulizing apparatus for medical use with improved nozzle positioning structure

(76) Inventor: Daniel C.M. Tseng, Taipei County (TW)

Correspondence Address: BIRCH STEWART KOLASCH & BIRCH, PO BOX 747, FALLS CHURCH, VA, 22040-0747, US

(21) Appl. No.: 10/882,278

(22) Filed: Jul. 2, 2004

(51) Int. Cl. A61M 11/00

(52) U.S. Cl. 128/200.14

(57) ABSTRACT

A nebulizing apparatus for medical use with a nozzle positioning structure, in which the direction of the nozzle can be adjusted and fixed according to the user. The nozzle and the nebulizing device are connected to each other by engagement protrusion and engagement grooves. The nozzle can be pulled out, rotated to a suitable angle and then installed on the cover of the nebulizing device if a different direction of the nozzle is desired.

### FIELD OF THE INVENTION

A nebulizing apparatus for medical use with improved nozzle positioning structure, and more particularly to a nebulizing apparatus for medical use with improved nozzle positioning structure having a nozzle and a nebulizing device connected to each other by means of engagement protrusions and engagement grooves.

### BACKGROUND OF THE INVENTION

In the prior arts, Taiwan Patent No. 567855 and U.S. Publication No. 2001/0002592 A1 disclosed an apparatus for nebulizing a liquid for medical use nebulizes a liquid medicine to fully generate the cure effect and includes a main body provided with an air compressor disposed therein, a nebulizing device to receive liquid medicine connected to the air compressor the liquid medicine, and an output device. The air compressor compresses the air in high speed and delivers the compressed air to the nebulizing device. The nebulizing device nebulizes the liquid medicine, and the output device outputs the nebulized liquid medicine for medical use.

In above-mentioned prior arts, the output device is disposed on the cover of the nebulizing device. The output device includes an input portion with smooth annular edge, and the cover of nebulizing device has a smooth annular groove for housing and centering the input portion of the output device. Therefore, when the output device is attached to the nebulizing device, it can rotate smoothly in the annular groove to change the direction of the output portion.

Nevertheless, since the output device can rotate smoothly on the nebulizing device, user often experiencing inconveniences when trying to fix the direction of the output device, which easily swiveling around the annular groove whenever it is touched incautiously.

### SUMMARY OF THE INVENTION

The objective of the present invention is to provide a nebulizing apparatus for medical use with improved nozzle positioning structure capable of adjusting and fixing the direction of the nozzle according to a user.

In order to achieve the foregoing objective, the present invention provides a nebulizing apparatus for medical use with improved nozzle positioning structure including a nozzle and a nebulizing device connected to each other by means engagement protrusions and engagement grooves. A cover of the nebulizing device is provided with a plurality of engagement grooves and lumpy portions disposed around the inner wall and outer wall of the cover. An input portion of the nozzle has an engagement protrusion which can be assembled corresponding to the inner wall engagement grooves of the cover to fix the position the nozzle. The nozzle can then be pulled out, rotates to suitable angle and then installed to the cover of the nebulizing device if a different direction of the nozzle is desired.

It is another objective of the present invention that the nozzle can be stored inside the protective cover to maintain the sanitary condition of the nozzle after usage of the nebulizing apparatus.

In order to achieve the foregoing objectives, the present invention discloses a protective cover having two fixing portions which are disposed on an inner wall of the protective cover and in accordance with the input portion of the nozzle, such that the input portion of the nozzle is embedded between the two fixing portions during installation and cannot be dropped. Then, the protective cover is disposed on the main body for further receiving the nozzle so as to save space and keep the nozzle clean.

### BRIEF DESCRIPTION OF THE DRAWINGS

These objectives shall become more readily apparent from the detailed description of preferred embodiment illustrated, purely by way of non limiting example, in the accompanying drawings, in which:

FIG. 1 is a perspective schematic view of a nebulizing apparatus for medical use with improved nozzle positioning structure of the present invention.

FIG. 2 is a perspective exploded schematic view of a nebulizing apparatus for medical use with improved nozzle positioning structure of the present invention.

FIG. 3 is a top plan schematic view of a nebulizing apparatus for medical use with improved nozzle positioning structure of the present invention.

FIG. 4 is a perspective schematic view showing an embodiment of a nebulizing apparatus for medical use with improved nozzle positioning structure of the present invention.

### DESCRIPTION OF THE PREFERRED EMBODIMENTS

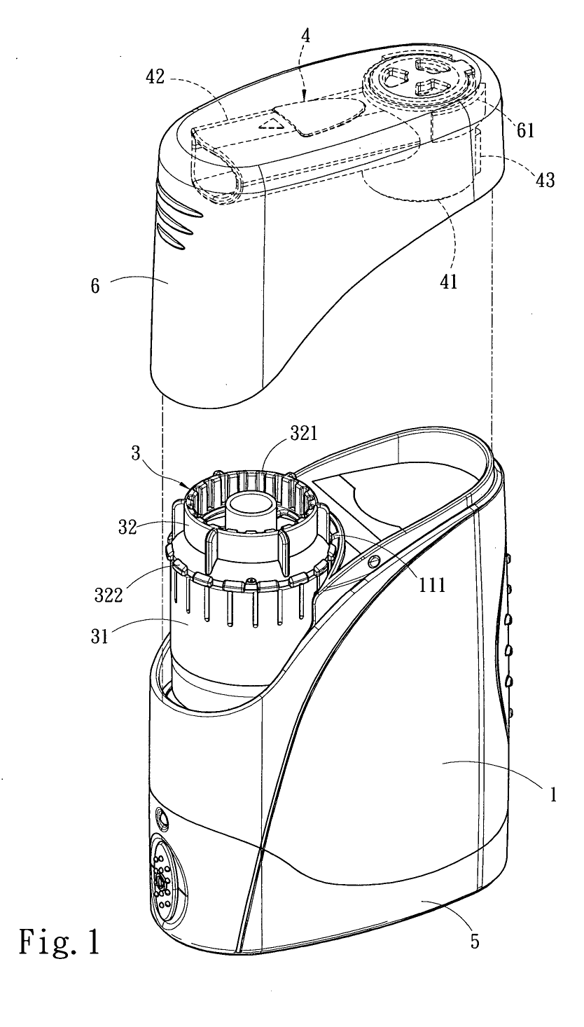

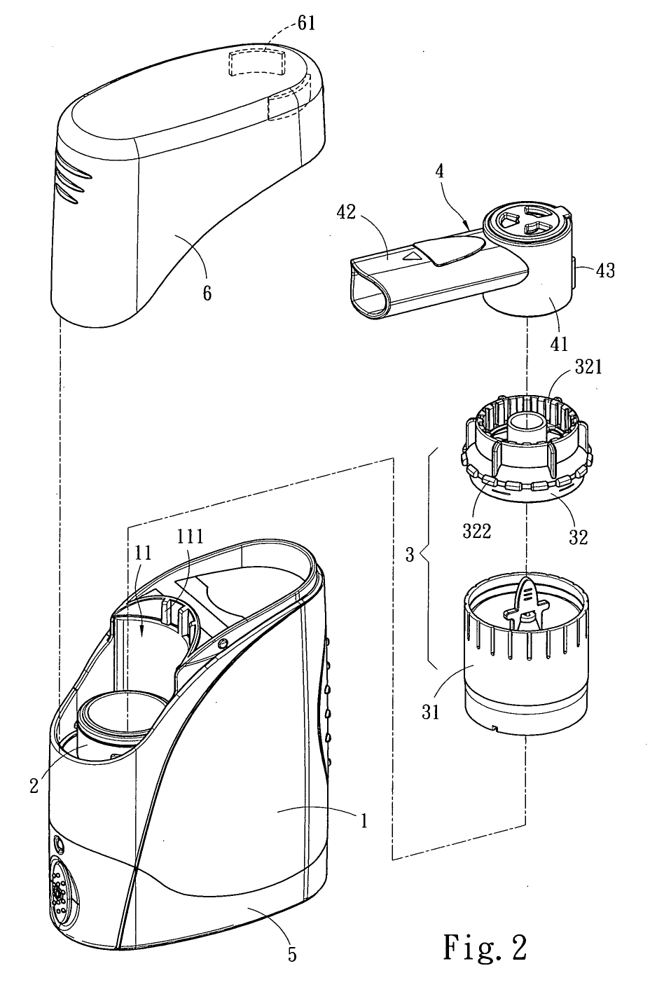

Referring to FIG. 1, it depicts a perspective schematic view of a nebulizing apparatus for medical use with improved nozzle positioning structure of the present invention. As shown in FIG. 1, the apparatus includes a main body 1 provided with an air compressor 2 disposed therein (shown in FIG. 2), a nebulizing device 3 to receive liquid medicine installed on the air compressor 2, a nozzle 4 installed on the nebulizing device 3, a bottom base 5 installed on a bottom of the main body 1, and a protective cover 6 installed on the main body 1 and receiving the nozzle 4. The air compressor 2 of the present invention compresses the air in high speed and transport the compressed air to the nebulizing device 3. The nebulizing device 3 nebulizes the liquid medicine, and the nozzle 4 outputs the nebulized liquid medicine for medical use.

Referring to FIG. 2, it depicts a perspective exploded schematic view of a nebulizing apparatus for medical use with improved nozzle positioning structure of the present invention. As shown in FIG. 2, the main body 1 has an open receiving space 11 for assemblage with the air compressor 2 and exposing the air compressor 2 for assemblage with the nebulizing device 3, wherein an inner wall of the receiving space 11 has at least one first engagement protrusion 111.

The nebulizing device 3 has a medicine cup 31 and a cover 32 disposed on the medicine cup 31, wherein the cover 32 is provided with a plurality of engagement grooves 321 disposed around an inner wall of the cover 32 and a plurality of lumpy portions 322 disposed around an outer wall of the cover 32, whereby the first engagement protrusion 111 of the receiving space 11 can be meshed into the lumpy portions 322 accordingly when the nebulizing device 3 is installed on the air compressor 2.

The nozzle 4 has an input portion 41 and an output portion 42, wherein the input portion 41 of the nozzle 4 has at least one second engagement protrusion 43 which can be meshed into the engagement grooves 321 accordingly during installation.

The protective cover 6 has two fixing portions 61 which are disposed on an inner wall of the protective cover 6 and corresponding with the input portion 41 of the nozzle 4, such that the input portion 41 is embedded between the two fixing portions 61 during installation.

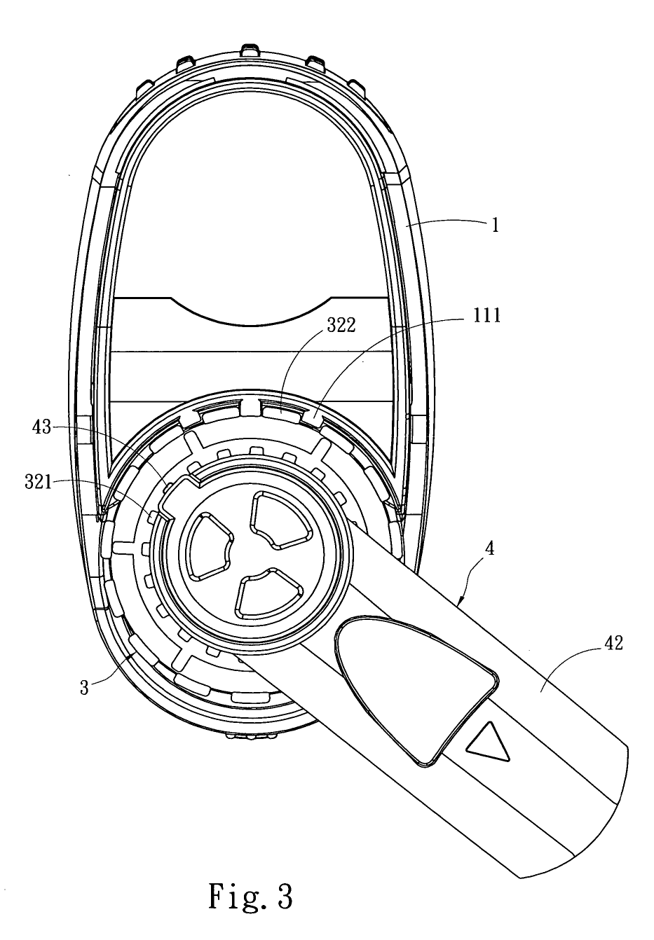

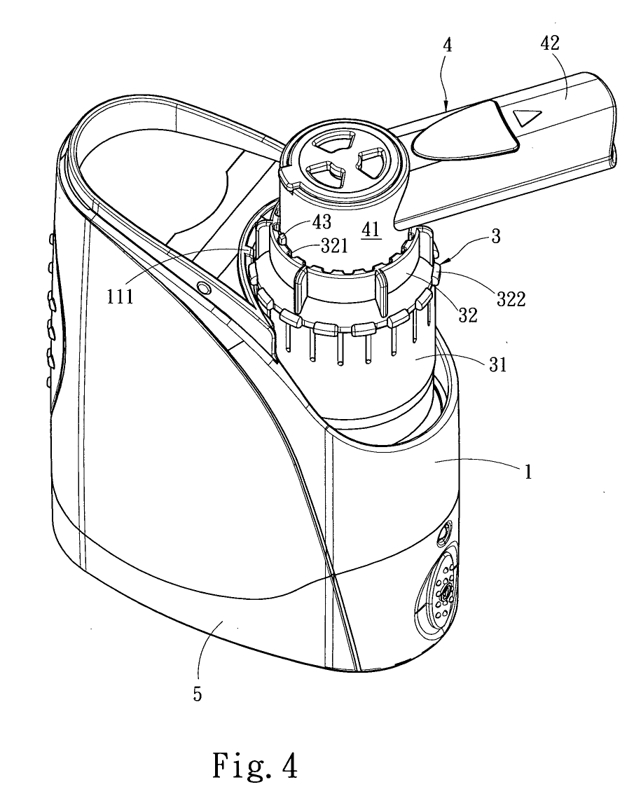

FIGS. 3 and 4 depict a top plan schematic view and a perspective schematic view showing an embodiment of the present invention. As shown in FIGS. 3 and 4, the nozzle 4 can be positioned by utilizing the engagement between engagement protrusion of the nozzle and engagement grooves of the cover, such that a user won't change the direction of the output portion 42 of the nozzle 4 during usage due to incautious touch. If the user wants to change the direction of the output portion 42, the user can pull the nozzle 4 and then install the nozzle 4 to the engagement grooves 321 of the cover 32 with different angle so as to meet the requirement of the user. Furthermore, the engagement between first engagement protrusion of the receiving space 11 and lumpy portions 322 of the cover will prevent undesired rotation of the cover on the medicine cup 31 during usage.

Although the invention has been explained in relation to its preferred embodiment, it is not used to limit the invention. It is to be understood that many other possible modifications and variations can be made by those skilled in the art without departing from the spirit and scope of the invention as hereinafter claimed.

1. A nebulizing apparatus for medical use with a nozzle positioning structure comprising:
a main body;
an air compressor disposed in the main body;
a nebulizing device installed on the air compressor and having a medicine cup and a cover disposed on the medicine cup;
a nozzle connected to the nebulizing device and having an input portion and an output portion; and
a protective cover installed on the main body;
wherein the nozzle is connected to the cover of the nebulizing device by engagement protrusions and engagement grooves, and a direction of the nozzle could be adjusted by pulling out the nozzle from the nebulizing device, rotating the nozzle to a suitable angle and then installing the nozzle on the cover of the nebulizing device, and
wherein the input portion of the nozzle has at least one second engagement protrusion and the cover is provided with a plurality of engagement grooves disposed around an inner wall of the cover, such that the second engagement protrusion is meshed into the engagement grooves during installation.

2. (canceled)

3. The apparatus according to claim 1, wherein the cover and the corresponding position on the main body has at least one engagement protrusion and engagement groove, such that the cover and the main body can be fixed by engagement protrusions and engagement grooves.

4. The apparatus according to claim 1, wherein the protective cover has two fixing portions disposed on an inner wall of the protective cover in accordance with the input portion of the nozzle, such that the nozzle is engaged between the two fixing portions.
